(19) United States
(12) Patent Application Publication
(10) Pub. No.: US 2026/0033853 A1
(43) Pub. Date: Feb. 5, 2026

(54) INTERATRIAL MULTI-CUSPID VALVULAR SHUNT

(71) Applicant: Medtronic, Inc., Minneapolis, MN (US)

(72) Inventors: Thomas A. Anderson, New Hope, MN (US); Zhongping Yang, Woodbury, MN (US); Neal C. Duong, Ramsey, MN (US); Nicolas Coulombe, Anjou (CA); Kevin R. Seifert, Forest Lake, MN (US); Kevin L. Sack, New Brighton, MN (US)

(21) Appl. No.: 19/125,150

(22) PCT Filed: Oct. 25, 2023
(86) PCT No.: PCT/US2023/077766

§ 371 Date: Apr. 28, 2025

Related U.S. Application Data
(60) Provisional application No. 63/381,284, filed on Oct. 27, 2022.

(51) Int. Cl. A61B 17/32; A61B 17/00; A61B 90/00

(52) U.S. Cl. CPC A61B 17/32; A61B 17/00234; A61B 90/08; A61B 2017/00252; A61B 2017/00292; A61B 2017/00557; A61B 2090/08021

(57) ABSTRACT

An example medical device includes an elongated support member defining a longitudinal axis and a plurality of expandable members at a distal portion of the elongated support member positioned circumferentially about the elongated support member. At least a portion of each of the expandable members is configured to radially extend from the elongated support member. Each expandable member of the plurality of expandable members comprises a respective cutting element, each cutting element comprising a cutting surface facing towards the longitudinal axis and configured to initiate a respective cut of septal wall tissue at a first end of the respective cut and to cut the septal wall tissue from the first end of the cut to a second end of the respective cut to form a multi-cuspid valvular shunt. The second end of the cut comprises an intersection of the plurality of cuts of the plurality of cutting elements.

This application claims the benefit of U.S. Provisional Patent Application 63/381,284, filed Oct. 27, 2022, the entire content of which is incorporated herein by reference.

### TECHNICAL FIELD

This disclosure generally relates to medical devices and, more particularly, to medical devices and associated techniques for forming shunts.

### BACKGROUND

Pulmonary edema (or “oedema”) is an excessive build-up of fluid in the lungs of a patient. Pulmonary edema may result from one or more conditions, including left atrial pressure elevation due to heart failure. A heart of a patient with heart failure may not efficiently pump blood, which may cause a pressure build-up in the left atrium and may cause fluid to be pushed into the lungs. Patients experiencing heart failure and pulmonary edema currently have limited treatment options.

Interatrial shunting is a technique to decompress the left or right atria in patients suffering from heart failure. During the procedure, a blood flow pathway is created between the right atrium and the left atrium such that blood flows between them. In a typical procedure, the septal wall separating the atria is cut with a puncturing device and a mechanical device such as a stent is left in place to prevent tissue overgrowth and to maintain the shunt.

### SUMMARY

The present disclosure describes systems, devices, and techniques for creating a fluid pathway, or shunt, between the left atrium and right atrium of a heart of a patient. The shunt can be used to, for example, treat patients having heart failure and/or pulmonary edema. Some proposals for shunting procedures may cut septal tissue in a radially outwards direction from a cutting tool to form the cuspids of the multi-cuspid valvular shunt, which results in variation and uncertainty in the length of each of the cuts and the overall geometry, symmetry, and/or shape of the multi-cuspid valvular shunt. This, in turn, results in variation and uncertainty in the performance of the multi-cuspid valvular shunt to act as a valve, for example, asymmetrically formed cuspids that were intended to be formed symmetrically may have reduced valve performance and may “leak,” allow increased backflow, and/or reduced ability to regulate pressure between the left atrium and right atrium causing a greater variation in “regulated” pressure and uncertainty in “regulated” pressure. For example, cutting radially outwards from the puncture site of a cutting tool causes and/or allows the cut portion septal tissue to “tent,” i.e., move in a distal or proximal direction, during the cutting. Additionally, cutting radially outwards from the puncture site of a cutting tool causes the tension of the septal tissue to change along the length of the cut during cutting. Additionally, if the longitudinal axis of the cutting tool is angled relative to the surfaces of the septal wall, cutting radially outwards from the puncture site by advancing or retracting the cutting tool in the proximal and distal directions results in cuts that may be shorter or longer than they otherwise would have been if the tool was not at an angle.

In examples described herein, techniques, systems, and devices include forming a multi-cuspid valvular shunt by initiating cutting of septal wall tissue at a first end of a cut that is radially outwards from an intersection of a plurality of cuts that form the multi-cuspids, and cutting radially inwards from the first end in a radially inwards direction to a second end of the cut that is at the intersection of the plurality of cuts.

Accordingly, the techniques, systems, and devices may provide one or more technical advantages that realize at least one practical application. For example, the techniques may reduce variation and uncertainty in the length of each of the cuts and improve the overall geometry, symmetry, and/or shape of the multi-cuspid valvular shunt. Tenting of septal wall tissue and variation of tension of the septal wall tissue along the cut may be reduced by initiating a cut at a position radially outwards from an intersection of cuts and cutting towards the intersection. Additionally, the length, uniformity, and straightness of a cut made towards the intersection may be substantially independent of variations of septal wall tissue properties along the cut, e.g., variations in the thickness and tension of the septal wall tissue. Further, initiating the cuts of a multi-cuspid valvular shunt at positions radially outwards from the intersection of the cuts establishes the length of each of the cuts and removes variation in the length of the cuts due to the cutting process. For example, if the longitudinal axis of the cutting tool is angled relative to the surfaces of the septal wall, initiating the cuts of a multi-cuspid valvular shunt at positions radially outwards from the intersection of the cuts predetermines the length of the cuts and reduces or eliminates variation in the lengths of the cuts due to cutting tool angle.

In some examples, the techniques, systems, and devices may include providing a distal and/or proximal support structure and/or backing material to a distal and/or proximal surface of the septal wall tissue. The distal and/or proximal support structure may be configured to prevent septal wall tissue from moving in the distal and/or proximal directions while cutting, and/or may reduce or prevent stretching of septal wall tissue along the cut while cutting, thereby reducing variation in the length, straightness, and width of the cut as well as reducing changing properties of the septal wall tissue at or near the cut, e.g., thickness, density, elasticity, or the like.

In one example, this disclosure describes a medical device including: an elongated support member defining a longitudinal axis; and a plurality of expandable members at a distal portion of the elongated support member, wherein the plurality of expandable members are positioned circumferentially about the elongated support member, wherein at least a portion of each of the expandable members is configured to radially extend from the elongated support member, wherein, to form a multi-cuspid valvular shunt, each expandable member of the plurality of expandable members comprises a respective cutting element of a plurality of elements, each cutting element of the plurality of cutting elements comprising a cutting surface facing towards the longitudinal axis and configured to initiate a respective cut of a plurality of cuts of a septal wall tissue at a first end of the respective cut and to cut the septal wall tissue from the first end to a second end of the respective cut comprising an intersection of the plurality of cuts.

In another example, this disclosure describes a method including: initiating a cut of a septal wall tissue between a right atrium and left atrium of a heart of a patient at a first end of the cut, wherein the cut comprises a second end at an intersection of two or more cuts of a multi-cuspid valvular shunt; and cutting the septal wall from the first end of the cut to the second end of the cut to form a portion of the multi-cuspid valvular shunt.

In another example, this disclosure describes a medical system including: a catheter defining a lumen; an inner member configured to be received in the catheter lumen and extend distally outward from a distal opening of the catheter, wherein the inner member comprises: an elongated support member configured to move axially within the catheter lumen, the elongated support member defining a longitudinal axis; and a plurality of expandable members at a distal portion of the elongated support member, wherein the plurality of expandable members are positioned circumferentially about the elongated support member, wherein at least a portion of each of the expandable members is configured to radially extend from the elongated support member, wherein, to form a multi-cuspid valvular shunt, each expandable member of the plurality of expandable members comprises a respective cutting element of a plurality of elements, each cutting element of the plurality of cutting elements comprising a cutting surface facing towards the longitudinal axis and configured to initiate a respective cut of a plurality of cuts of a septal wall tissue at a first end of the respective cut and to cut the septal wall tissue from the first end to a second end of the respective cut comprising an intersection of the plurality of cuts.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a perspective view depicting an example medical system configured to form a shunt at a target treatment site.

FIG. 2A is a schematic diagram illustrating an example inner member including a distal portion in an undeployed configuration.

FIG. 2B is a schematic diagram illustrating an example inner member including a distal portion in a deployed configuration.

FIG. 3A is a schematic diagram illustrating an example inner member including a support structure in an undeployed configuration and a distal portion in a deployed configuration.

FIG. 3B is a schematic diagram illustrating an example inner member including a support structure in a deployed configuration and distal portion in a deployed configuration.

FIG. 4A is a cross-sectional schematic view depicting an example interatrial multi-cuspid valvular shunt.

FIG. 4B is a cross-sectional schematic view depicting another example interatrial multi-cuspid valvular shunt.

FIG. 4C is a cross-sectional schematic view depicting another example interatrial multi-cuspid valvular shunt.

FIG. 4D is a cross-sectional schematic view depicting another example interatrial multi-cuspid valvular shunt.

FIG. 4E is a cross-sectional schematic view depicting another example interatrial multi-cuspid valvular shunt.

FIG. 5 is a flow diagram illustrating an example technique for forming a biostable, multi-cuspid valvular shunt between the left and right atria of a heart of a patient.

FIG. 6A is a schematic diagram illustrating penetrating a septal wall with an example medical system.

FIG. 6B is a schematic diagram illustrating extending a cutting tool of an example medical system in preparation for cutting a septal wall.

FIG. 6C is a schematic diagram illustrating cutting a septal wall with a cutting tool of an example medical system.

FIG. 7 is a schematic perspective view depicting an example medical system including an example deployment mechanism.

### DETAILED DESCRIPTION

The disclosure describes examples of medical systems, devices, and techniques for creating a fluid pathway, or shunt, between the left atrium and right atrium of a heart of a patient without the use of an implant, e.g., such as a stent to maintain the shunt. The shunt may be formed as a valve, e.g., a multi-cuspid valve, in a septal wall between the left and right atrium. Example techniques, systems, and devices include forming a multi-cuspid valvular shunt by initiating cutting of septal wall tissue at a first end of a cut that is radially outwards from an intersection of a plurality of cuts that form the multi-cuspids, and cutting radially inwards from the first end in a radially inwards direction to a second end of the cut that is at the intersection of the plurality of cuts.

The techniques of this disclosure can be used to treat heart failure, pulmonary edema, or the like. For instance, forming a shunt between the left atrium and the right atrium with the systems and devices described herein enable the relief of fluid build-up in the lungs of a patient without requiring the permanent implantation of a foreign object (e.g., a stent or the like), leading to better patient outcomes. For example, the techniques described herein may reduce pressure in the left atrium and benefit left ventricular loading, both of which may lead to better patient outcomes and reductions in hospitalizations. In addition, the systems and devices described herein are highly user-friendly, e.g., do not require extensive training for the clinician.

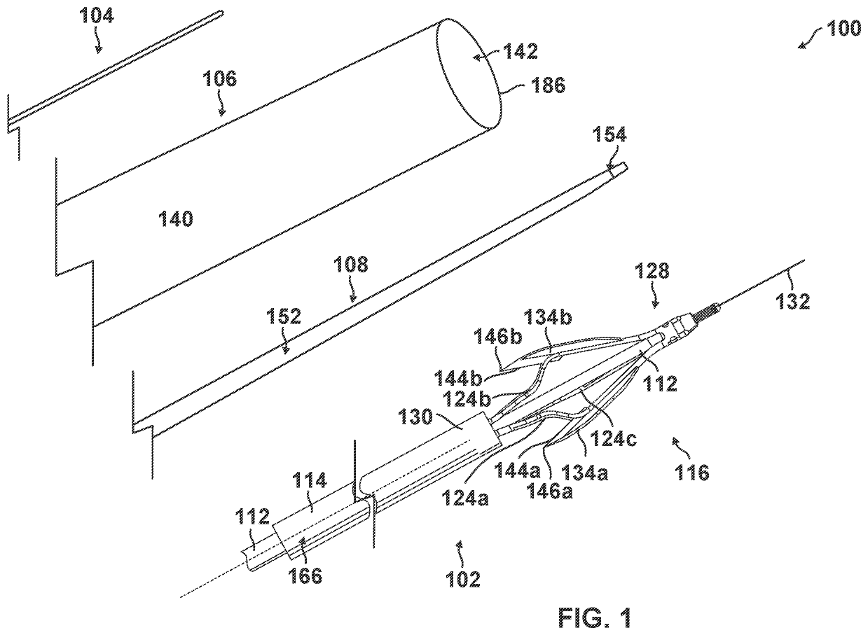

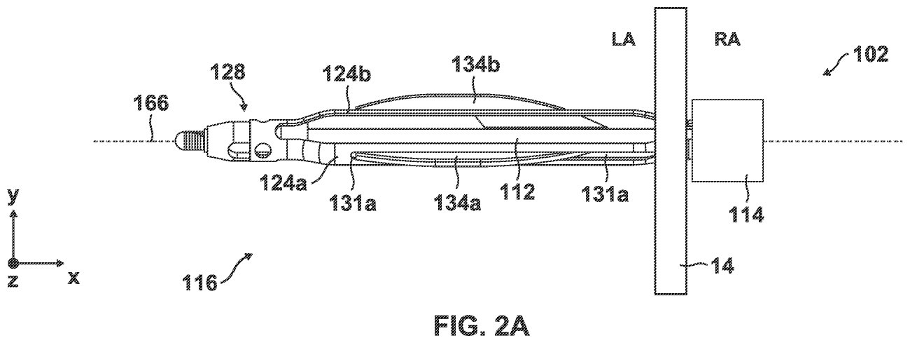

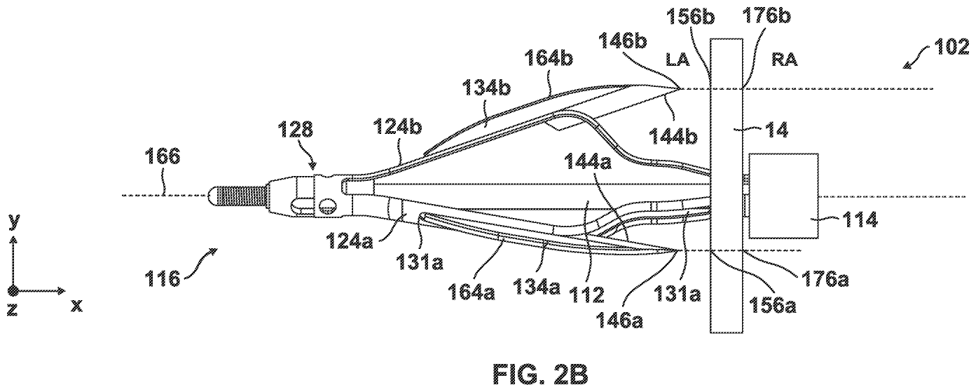

FIG. 1 is a perspective view depicting an example medical system 100 configured to form a shunt at a target treatment site, such as, for example, between a left atrium and a right atrium of a heart of a patient. FIG. 2A is a schematic diagram illustrating an example inner member 102 including a distal portion 116 in an undeployed configuration (e.g., a delivery configuration), and FIG. 2B is a schematic diagram illustrating the example inner member 102 including the distal portion 116 in a deployed configuration. In the examples shown in FIGS. 1-3, medical system 100 includes inner member 102, which may be an example of a cutting tool. In some examples, but not all examples, medical system 100 also includes a separate puncturing tool 108. In other examples, puncturing tool 108 may be part of inner member 102 or a different device. Medical system 100 is also shown in FIG. 1 as including a guidewire 104, and a catheter 106.

In the examples shown in FIGS. 1-3, inner member 102 includes an elongated support member 112, a movable member 114, and a distal portion 116. In some examples, elongated support member 112 defines a device inner lumen configured to receive, e.g., a guidewire 104 and/or puncturing tool 108. Guidewire 104 can, for example, be used to help navigate inner member 102 through vasculature of a patient to a target treatment site within the patient. Elongated member 112 is at least partially within a lumen of movable member 114, and inner member 102 is configured to be movable within a lumen 142 of catheter 106.

Distal portion 116 is coupled to elongated support member 112, e.g., at attachment portion 128 at a distal end of distal portion 116 and a distal end of elongated support member 112 as shown. Distal portion 116 is coupled to movable member 114, e.g., at attachment portion 130 at a proximal end of distal portion 116 and a distal end of movable member 114 as shown. In some examples, as detailed further below, one or both of movable member 114 and elongated support member 112 are configured to be longitudinally translatable along a longitudinal axis 166 defined by elongated support member 112 to change a configuration of distal portion 116, e.g., between an undeployed configuration and a deployed configuration. For example, distal portion 116 includes a plurality of expandable members 124a, 124b, and 124c (collectively, “expandable members 124,” noting expandable member 124c is obstructed by elongated support member 112 and not visible in the examples shown) positioned circumferentially about elongated support member 112. At least a portion of each of the plurality of expandable member 124 is configured to radially extend and/or move in the radial direction to and from elongated support member 112, e.g., upon compression and/or tension of the plurality of expandable members due to longitudinal translation of movable member 114 relative to elongated support member 112.

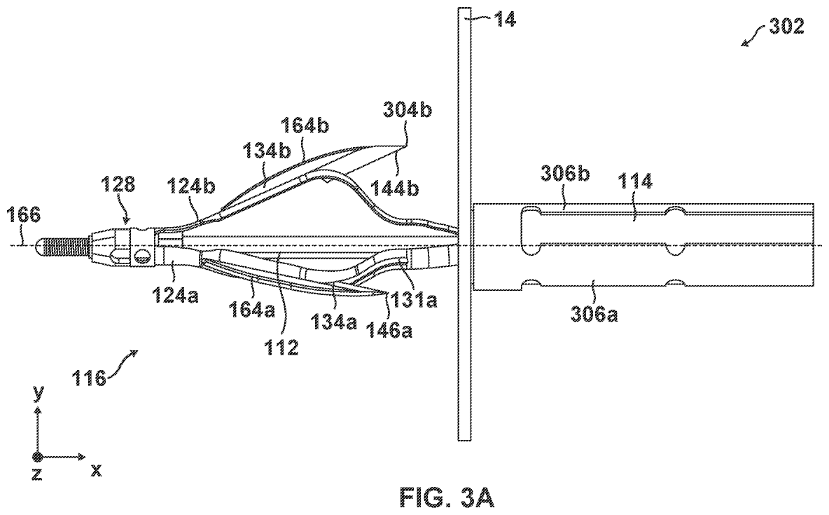

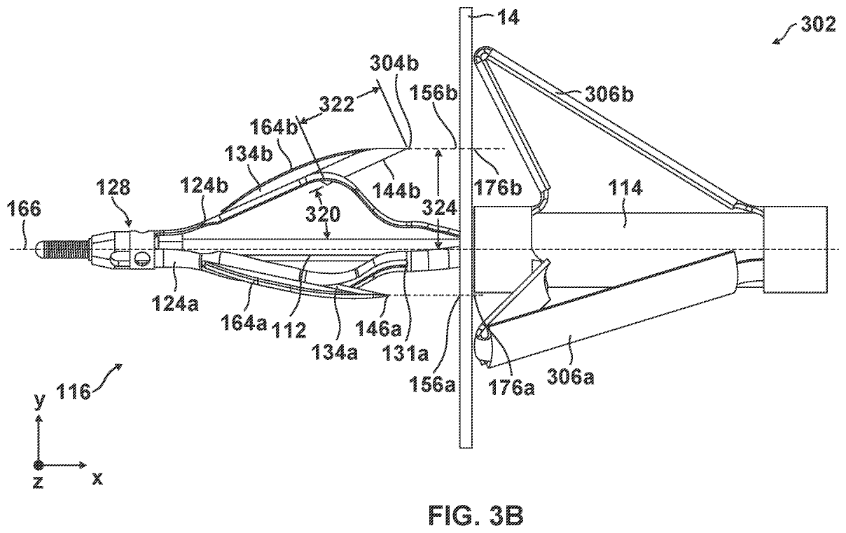

In the examples shown in FIGS. 1 and 3, expandable members 124 are in a deployed configuration in which a portion (e.g., the non-attached portions) of each of expandable members 124 extend radially away from elongated support member 112, e.g., expandable members 124 are “expanded” radially. To change distal portion 116 to the deployed configuration, elongated support member 112 and movable member 114 may be longitudinally translated relative to each other such that attachment portions 128 and 130 are moved nearer to each other compressing expandable members 124 and causing the expandable members 124 to move radially away from elongated support member 112. To change distal portion 116 to an undeployed configuration, a user may translate elongated support member 112 and movable member 114 relative to each other such that attachment portions 128 and 130 are moved farther from each other and exerting tension on expandable members 124 and causing the expandable members 124 to move radially towards elongated support member 112. In the undeployed configuration, expandable members 124 may be substantially adjacent to and substantially straight along an outer surface of elongated support member 112. In other words, in the undeployed configuration the expandable members 124 may be configured to be retracted so as to fit and be movable within lumen 142 of catheter 106. Each of expandable members 124 may be coupled and/or attached to movable member 114 and elongated support member 112 as described above relative to distal portion 116.

In other examples, expandable member124 may be formed with a spring bias configured to extend and/or move radially away from elongated support member 112, e.g., in the absence of compression and/or tension due to movement of attachment portion 128 and 130. For example, expandable members 124 may comprises Nitinol that is heat set such that expandable members 124 and distal portion 116 is in the deployed configuration in a relaxed, or default, state in which tension or compression is not applied to expandable members 124 via attachment portion 128 and 130. In some examples, to change distal portion 116 to an undeployed configuration, a user may translate elongated support member 112 and movable member 114 relative to each other such that attachment portions 128 and 130 are moved farther from each other and exerting tension on expandable members 124 and causing the expandable members 124 to move radially towards elongated support member 112, as described above. In other examples, to change distal portion 116 between delivery and deployed configurations, a user may translate a sheath (not shown) proximally or distally to cause the expandable members 124 to move radially towards elongated support member 112 or to release expandable members 124 to move radially away from elongated support member 112.

In some examples, elongated support member 112 includes an atraumatic distal tip or distal portion, e.g., formed from a relatively soft polymer material (not shown). In some examples, a distal guidewire 132, such as a Nitinol wire or another elongated guide member, extends distally outward from a distal-most end of elongated support member 112. Guidewire 132 can be, for example, embedded in elongated support member 112 or extend through a lumen defined by elongated support member 112 and extend distally outward from a distal mouth or opening of elongated support member 112. In some examples, guidewire 132, in addition to, or instead of, puncturing tool 108, is configured to function as a puncturing element configured to puncture through tissue, e.g., septal wall tissue, of a patient to enable advancement of at least distal portion 116 and elongated support member 112 through the tissue. In some examples, guidewire 132 may be a conductor and may be configured to be electrified and/or heated.

Expandable members 124 may be made of a metal, a plastic, or any suitable material with sufficient stiffness to support cutting members 134 to cut tissue of a patient and sufficient flexibility and/or elasticity to expand and contract radially in response to longitudinal compression and tension, as described herein. In some examples, expandable members 124 may be made of Nitinol. Although illustrated and described as having three expandable members 124a, 124b, and 124c, distal portion 116 may include fewer or more expandable members 124, e.g., one expandable member 124, two expandable members 124, or four or more expandable members 124.

In the examples shown, expandable members 124 each include cutting members 134 configured to cut tissue of the patient, e.g., septal wall tissue. For example, expandable member 124a includes cutting member 134a, expandable member 124b includes cutting member 134b, and expandable member 124c includes cutting member 134c (not shown). Cutting members 134a, 134b, and 134c (collectively “cutting members 134”) may be made of a metal, a plastic, or any suitable material for cutting tissue of the patient. In some examples, cutting members 134 may be blades, e.g., formed as razor blades having a razer and/or very thin and relatively hard cutting edges 144a, 144b, and 144c (collectively, “cutting edges 144,” cutting edge 144c not shown). In the examples shown, cutting members 134 are attached to expandable members 124, e.g., via mounting slots 131 illustrated in FIGS. 2A and 2B. In the example shown, cutting members 134 are positioned on a proximal portion of expandable members 124 such that in the deployed configuration (as shown), the cutting edges 144 of cutting members 134 are angled towards longitudinal axis 166, e.g., cutting surfaces 144 are facing towards longitudinal axis 166 and inner member 102 is configured to cut septal wall tissue when moved proximally, or “drawn back,” with expandable members 124 in the deployed configuration.

For example, cutting members 134 may include respective cutting tips 146, e.g., cutting member 134a may including cutting tip 146a, cutting member 134b may including cutting tip 146b, cutting member 134c may including cutting tip 146c (not shown), and cutting tips 146a, 146b, and 146c (collectively, “cutting tips 146”) may be configured to initiate a cut in septal wall 14 tissue. For example, cutting tip 146a may be configured to initiate a cut in septal wall 14 at position 156a, cutting tip 146b may be configured to initiate a cut in septal wall 14 at position 156b, and cutting tip 146c (not shown) may be configured to initiate a cut in septal wall 14 at a position 156c (not shown). Each of positions 156a, 156b, and 156c (collectively, “positions 156”), may be located radially away from an intersection position in septal wall 14, where the intersection position is the position at which a plurality of cuts in septal wall 14 tissue made by inner member 102 and cutting members 134 meet, e.g., at longitudinal axis 166 in the examples shown. In other words, to form a multi-cuspid valvular shunt in septal wall 14, cutting members 134 are configured to initiate respective cuts of septal wall 14 tissue at a first position or “end” of a cut, e.g., positions 156, and cutting members 134 may be configured to then cut septal wall 14 tissue from the respective first ends of the cuts, e.g., positions 156, in a radially inwards direction (e.g., substantially in the y-z plane corresponding to the plane of the portion of septal wall 14 shown in FIG. 2B) to respective second ends of the cuts which may be an intersection point of two or more of the cuts, e.g., the position on septal wall 14 at which longitudinal axis 166 intersects with septal wall 14, or in other words, the position at which medical system 100 punctures septal wall 14, and in some examples, the position comprising a radially central portion of the multi-cuspid valvular shunt.

In the example shown in FIG. 2A, septal wall 14 is punctured at the intersection between septal wall 14 and longitudinal axis 166, and distal portion 116 of inner member 102 is advanced distally (e.g., in the negative x-direction as shown) through septal wall 14 from the right atrium RA to the left atrium LA with distal portion 116 in an undeployed configuration. In the example shown in FIG. 2B, distal portion 116 is in the left atrium LA and in the deployed configuration in which expandable members 124, including cutting elements 134, are expanded radially outwards from elongated support member 112 and longitudinal axis 166. Distal portion 116 and cutting elements 134 are in position to be retracted proximally to initiate cuts of septal wall 14, e.g., via contacting at least one of cutting tips 146 or cutting surfaces 144 to septal wall 14 at positions 156. Distal portion 116 and cutting elements 134 may then be further retracted to cut septal wall from the first ends of cuts, e.g., at positions 156, to the second ends of the cuts, e.g., at the puncture in septal wall 14 and an intersection of the cuts, which may also be at or near the intersection of longitudinal axis 166 with septal wall 14 and a radially central portion of a multi-cuspid valvular shunt in septal wall 14.

In some examples, each of cutting elements 134 is configured to determine a respective length of a respective cut of a plurality of cuts that form a multi-cuspid valvular shunt by initiating the respective cut at positions 156 that are radially outwards from the intersection of the plurality of cuts. In some examples, initiating the plurality of cuts at positions 156 radially outwards from the intersection of the cuts causes the respective lengths of the respective cuts to be independent of thickness variations of septal wall 14, density and/or durometer variations of septal wall 14, tension of septal wall 14, and/or an angle of distal portion 116 and/or inner member 102 relative to a surface of septal wall 14. For example, once cutting tips 146 contact septal wall 14 and initiate cuts at positions 156, the length of the cuts are determined by the distance from the intersection of the plurality of cuts and positions 156. If inner member 102 is at an angle with respect to septal wall 14, or if the material properties of septal wall 14 change along the length of the cuts, the distance between the intersection of the cuts (e.g., at about where inner member 102 has punctured septal wall 14) and positions 156 does not change. In the example shown, cutting elements 134 are at an angle relative to longitudinal axis 166 with cutting surfaces facing longitudinal axis 166 (e.g., cutting surfaces are at less than a 90 degree angle with respect to longitudinal axis 166) such that retracting inner member 102 in the proximal direction (the positive x-direction) results in cutting septal wall 14 from positions 156 to an intersection point of the cuts, namely, at or about where inner member 102 has punctured septal wall 14 and/or the intersection of longitudinal axis 166 and septal wall 14.

In some examples, expandable members 124 and cutting elements 134 may be configured to be retracted radially inwards towards longitudinal axis 166 to cut septal wall 14 from positions 156 to the second ends of the cuts, e.g., to the intersection of the cuts. For example, expandable members 124 may be configured to cause cutting elements to move radially inwards towards longitudinal axis 166 to cut septal wall 14 in a slicing or scissoring-like action. In some examples, expandable members 124 and cutting elements 134 may be configured to be retracted radially inwards while retracting distal portion 116 proximally, e.g., towards the proximal side of septal wall 14 to cut septal wall 14 via both moving angled cutting surfaces 144 distally and “scissoring” cutting elements 134 by moving cutting elements 134 radially inwards towards longitudinal axis 166. In some examples, inner member 102 and expandable members 124 are configured to initiate a plurality of cuts substantially concurrently, e.g., via retracting inner member 102 proximally to contact cutting point 146 and/or cutting surfaces 144 to septal wall 14 at a plurality of positions 156 (e.g., 156a, 156b, and 156c), and inner member 102 and expandable members 124 are configured to cut each of the plurality of cuts substantially concurrently.

In some examples, expandable members 124 may be configured to prevent cutting tips 146 and/or cutting surfaces 144 from contacting tissue of the patient when expandable members 124 are radially retracted to elongated support member 112, e.g., in the undeployed configuration. For example, expandable member 124 include slots 131 which may be configured to receive at least cutting tips 146 and/or cutting surfaces 144 such that at least cutting tips 146 and/or cutting surfaces 144 are not exposed to tissue of the patient, as shown in FIG. 2A. In some examples, slots 131 may be configured or receive the entirety of cutting elements 134 such that none of cutting elements 134 are exposed to patient tissue in the undeployed configuration, and expandable members 124 may have substantially atraumatic and/or smooth surfaces. In other examples, a portion of cutting elements 134 may not be received within slots 131, and such portions of cutting elements 134 may have smooth and/or atraumatic surfaces. For example, cutting elements 134 may have an atraumatic, smooth, and/or non-cutting surfaces 164 (e.g., 164a, 164b, and 164c (not shown)) as shown in FIGS. 2A-2B.

In some examples, cutting members 134 may be positioned on a distal portion of expandable members 124 such that in the deployed configuration, the cutting edges 144 of cutting members 134 are angled towards the distal direction, e.g., inner member 102 is configured to cut septal wall tissue when moved distally, or “pushed forward,” with expandable members 124 in the deployed configuration. For example, inner member 102 may not penetrate septal wall 14 before cutting, and distal portion 116 may be configured to be in the deployed configuration in the right atria RA. Inner member 102 may then be moved distally such that cutting tips 146 and/or cutting surfaces 144 initiate cuts at positions 176a, 176b, and 176c (not shown), e.g., collectively “positions 176.” Positions 176 may be substantially the same as positions 156 described above, except that cutting positions 176 are on the proximal side (in the right atrium RA as shown) of septal wall 14, and cutting positions 156 are on the distal side (in the left atrium LA as shown) of septal wall 14. Inner member 102, expandable members 124, and cutting elements 134 may then initiate cuts and make cuts with, or without, scissoring action as described above, except that inner member 102 may be moved distally rather than proximally to initiate and make cuts.

Referring now to FIGS. 3A-3B, FIG. 3A is a schematic diagram illustrating an example inner member 302 including a support structure 306 in an undeployed configuration and a distal portion 116 in a deployed configuration, and FIG. 3B is a schematic diagram illustrating the example inner member 302 including the support structure 306 in a deployed configuration and distal portion 116 in a deployed configuration.

In some examples, support structures 306a, 306b, and 306c (not shown) (collectively “support structures 306) are configured to provide support to septal wall 14 during initiation of the cuts and during cutting of septal wall 14 with cutting elements 134. In the example shown, inner member 302 includes “distal” support structures 306 configured to support a distal surface of septal wall 14 during initiation and cutting, e.g., on the left atrium LA side illustrated in FIGS. 3A and 3B. In other examples, inner member 302 may include “proximal” support structures (not shown) configured to support a proximal surface of septal wall 14 during initiation and cutting, e.g., on the right atrium RA side illustrated in FIGS. 3A and 3B.

Support structures 306 (or proximal support structures, in some examples) may provide support of at least a portion of septal wall 14 along the length of each of the plurality of cuts and may be configured to reduce movement of the at least the portion of septal wall 14 in the proximal and/or distal directions, respectively, while cutting tips 146 and/or cutting surfaces 144 are cutting septal wall 14. In some examples, the proximal and/or distal support structures may comprise a backing (not shown) providing support to the entire area including the cut and areas adjacent to the cut. In the examples shown, support structures 306 comprise a structure providing support to areas adjacent to the cuts, e.g., but not the area of the cut such that cutting members 134 cut septal wall 14 without contacting support structures 306. In some examples, support structures 306 may be configured to reduce a stretching of the septal wall along the cuts while cutting tips 146 and/or cutting surfaces 144 are cutting septal wall 14. For example, the proximal and/or distal support structures may be configured to hold tissue of the septal wall to improve cutting elements 134 cutting through, or shearing, septal tissue with reduced dragging of septal tissue in the cutting direction. In some examples, the proximal and/or distal support structures may comprise an expandable balloon (not shown), an expandable frame, one or more expandable members which may or may not include a slots configured to receive cutting elements 134 after having cut through septal wall 14, or the like.

In the example shown, distal portion 116 includes cutting members 134 that are positioned on a proximal portion of expandable members 124 such that in the deployed configuration (as shown), the cutting edges 144 of cutting members 134 are angled at angle 320 towards longitudinal axis 166, e.g., cutting surfaces 144 are facing towards longitudinal axis 166 and inner member 102 is configured to cut septal wall tissue when moved proximally, or “drawn back,” with expandable members 124 in the deployed configuration. In some examples, angle 320 may be from about 15 degrees to about 40 degrees, or from about 20 degrees to about 30 degrees, or may be about 24 degrees. In some examples, cutting surfaces 144 may have a length 322 that is from about 3 millimeters (mm) to about 6, or from about 4 mm to about 5 mm, or from about 4.6 to 4.7 mm, or that may be about 4.67 mm. In some examples, in the deployed configuration, cutting tips 146 may extend radially away from longitudinal axis by a distance 324 that is from about 3 mm to about 8 mm, or from about 4 mm to about 7 mm, or from about 5.5 mm to about 6.5 mm, or that may be about 5.3 mm.

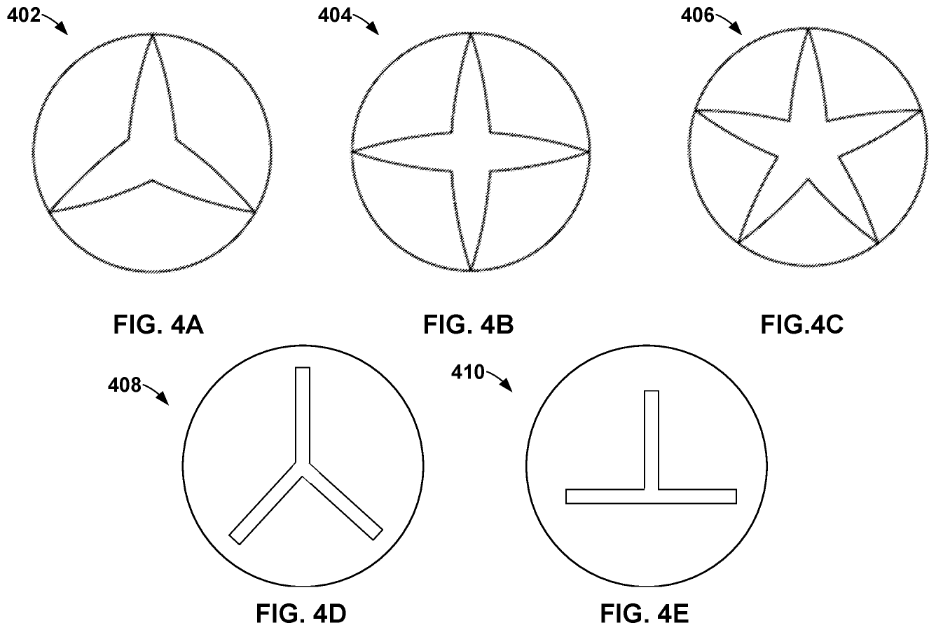

Referring now to FIGS. 4A-4E, FIG. 4A is a cross-sectional schematic view depicting an example interatrial tri-cuspid valvular shunt 402, FIG. 4B is a cross-sectional schematic view depicting an example interatrial quadri-cuspid valvular shunt 404, FIG. 4C is a cross-sectional schematic view depicting an example interatrial penta-cuspid valvular shunt 406, FIG. 4D is a cross-sectional schematic view depicting an example interatrial Y-cuspid valvular shunt 408, and FIG. 4E is a cross-sectional schematic view depicting an example interatrial T-cuspid valvular shunt 410. In some examples, inner member 102 is configured to form a multi-cuspid valvular shunt in septal wall 14 tissue of any one of valvular shunts 402-410. For example, inner member 102 including three expandable members 124 as illustrated in FIGS. 1-3 is configured to cut tri-cuspid valvular shunt 402. In other examples, inner member 102 may include four expandable members 124 and may be configured to cut quadri-cuspid valvular shunt 404, inner member 102 may include five expandable members 124 and may be configured to cut penta-cuspid valvular shunt 406, inner member 102 may include three expandable members 124 and may be configured to cut Y-cuspid valvular shunt 408, or inner member 102 may include at least two expandable members 124 and may be configured to cut T-cuspid valvular shunt 410.

Referring back to FIGS. 1-3, catheter 106 is configured to facilitate delivery of inner member 102, e.g., distal portion 116, to a target treatment site in a patient. Catheter 106 includes an elongated tubular body 140 defining a catheter inner lumen 142 and opening 186 to inner lumen 142. In some examples, elongated tubular body 140 and/or catheter 106 may be an introducer, or a deflectable introducer.

Medical system 100 includes a puncturing element configured to form an initial puncture through septal wall tissue. For example, the puncturing element can have an incisive tip configured to cut a pathway through tissue of a patient and/or another type of tip configured to define the pathway through tissue. In some examples, but not all examples, the puncturing element includes a distinct puncturing tool 108, which is physically separate from inner member 102. In other examples, the puncturing element may be part of inner member 102, such as the distal guidewire 132 (e.g., a Nitinol flat wire) extending from an atraumatic distal tip of elongated support member 112 of inner member 102.

As shown in FIG. 1, puncturing tool 108 includes an elongated structure 152, such as a guidewire, a hypotube, a catheter body, or the like, and an electrifiable distal tip 154, which is configured to electrically heat to facilitate the forming of a puncture through septal wall tissue. For instance, the electrifiable distal tip 154 may include a plasma electrode. In other examples, distal tip 154 is a relatively sharp incisive tip facilitating puncture through purely mechanical means.

In some examples, puncturing tool 108 further includes a dilation element (not shown), which is configured to expand radially outward to expand a puncture formed by puncturing tool 108. In some examples, the diameter of puncturing tool 108 may increase in a proximal direction from the distal end of puncturing tool 108 to dilate an initial puncture, e.g., puncturing tool 108 may have a tapered tip configured to dilate the initial puncture such as when puncturing tool is distally advanced through the puncture. In other examples, after distal tip 154 forms an initial puncture through septal wall tissue and the dilation element may be at least partially advanced through the puncture and expanded radially outward to dilate the puncture (forming a dilated puncture). The dilated puncture facilitates subsequent advancement of distal portion 116 of inner member 102 through the septal wall of the patient's heart. For example, the initial puncture formed by distal tip 154 may not be large enough to enable distal portion 116 to extend through the puncture.

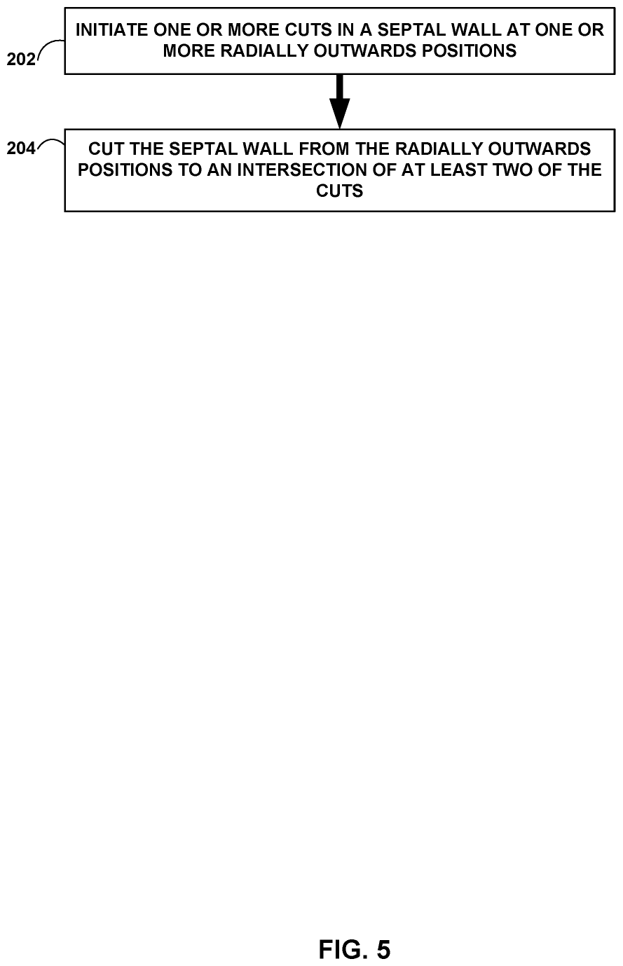

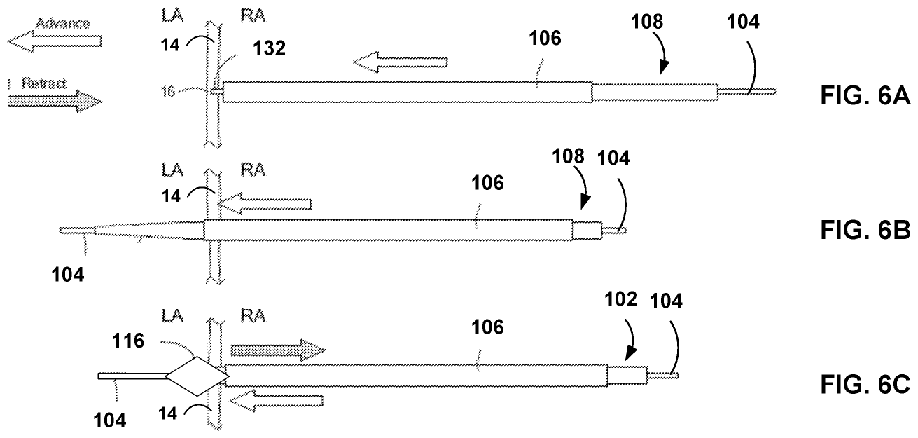

FIG. 5 is a flow diagram illustrating an example technique for forming a multi-cuspid valvular shunt between the left and right atria of a heart of a patient. FIGS. 6A-6C are schematic diagrams illustrating steps of the method of FIG. 5 using medical system 100 described above. The example technique of FIGS. 5-6C is described with reference to medical system 100, however, the example technique may be performed using any system including a device and/or tool including the functionality of inner member 102 described herein. The technique of FIGS. 5-6C may be performed by any suitable user, such as a cardiologist or other clinician.

A clinician may initiate one or more cuts of septal wall 14 tissue between a right atrium RA and left atrium LA of a heart of a patient at one or more radially outwards positions from an intersection point of the one or more cuts forming a multi-cuspid valvular shunt (202). For examples, the clinician may initiating a cut of septal wall 14 tissue between a right atrium RA and left atrium LA of a heart of a patient at a first end of the cut, where the cut includes a second end at an intersection of two or more cuts of the multi-cuspid valvular shunt. In some examples, the intersection comprises a radially central portion of the multi-cuspid valvular shunt.

For example, prior to initiating the one or more cuts, the clinician may puncture septal wall 14, and the clinician may advance catheter 106 to septal wall 14 between left and right atria of a patient's heart, e.g., through the femoral vein of the patient to access the right atrium. In some examples, the clinician may advance catheter 106 together with guidewire 104 to septal wall 14 between left and right atria. The clinician may advance puncture tool 108 with guidewire 104 and/or inner member 102 with guidewire 132 through lumen 142 of catheter 106 to septal wall 14. The clinician may then advance puncture tool 108 and/or inner member 102 into septal wall 14, and optionally electrify distal tip 154 or guidewire 132, to puncture septal wall 14 and form an opening 16 in septal wall 14 (FIG. 6A). In other examples, the clinician may advance guidewire 104 into septal wall 14, optionally electrify at least a distal portion of guidewire 104, to puncture septal wall 14 and form the opening 16, e.g., before or after insertion of and advancing inner member 102 to septal wall 14 through lumen 142. In some examples, the clinician may puncture septal wall 14 via RF ablation. In some examples, the clinician may use distal tip 154, guidewire 104, or guidewire 132 to pace the right atrium to locate the Fossa Ovalis for a target area to create an opening 16 in septal wall 14. For example, distal tip 154, guidewire 104, or guidewire 132 may use unipolar pacing RF energy to acquire electrogram signals to locate the Fossa Ovalis. When the Fossa Ovalis (or other target area) is located, the clinician may use the same distal tip 154, guidewire 104, or guidewire 132 to use ablative RF energy to puncture septal wall 14 to create opening 16, as shown in FIG. 6A. In some examples, the clinician may dilate opening 16, e.g., via a separate dilator tool, using puncture tool 108, or using inner member 102.

The clinician may advance inner member 102 through the septal wall. For example, the clinician may retract and/or remove puncture tool 108 from catheter 106, if used, and advance inner member 102 through lumen 142 of catheter 106 with distal portion 116 and expandable members 124 in an unexpanded, or delivery, configuration. The clinician may advance distal portion 116 through the puncture/opening in the septal wall, e.g., from the right atrium RA to the left atrium LA, as shown in FIG. 6B.

The clinician may cause expandable members 124 to expand radially away from elongated support member 112, and retract inner member 102 in the proximal direct towards septal wall 14 to initiate the cuts (FIG. 6C), e.g., at radially outwards positions 156 (FIGS. 2A-2B).

In some examples, initiating the cuts at the first ends of the cuts (e.g., positions 156) determines the lengths of the cuts. In some examples, initiating the cuts at the first ends of the cuts causes the lengths of the cuts to be independent of at least one of a thickness variation of septal wall 14, a density variation of septal wall 14, a tension of septal wall 14, or an angle of inner member 102 used to make the cuts, the angle being between the longitudinal axis 166 of inner member 102 and a surface of septal wall 14. For example, by way of contrast, if cuts are initiated at the second ends, e.g., the intersection point of the cuts, the lengths of the cuts may depend on one or more of a thickness variation of septal wall 14, a density variation of septal wall 14, a tension of septal wall 14, or an angle of inner member 102 used to make the cuts, the angle being between the longitudinal axis 166 of inner member 102 and a surface of septal wall 14, because the locations of the first ends, e.g., positions 156, are not determined or set. If inner member 102 is at an angle, cutting from the intersection and radially outwards would cause some cuts to be longer than others because of the angle. If the material properties of septal wall 14 change along the length of the cut when cutting from the intersection and radially outwards, septal wall tissue may require greater or less force to shear and separate the septal wall tissue to make the cuts, and the tissue may drag and/or stretch rather than separate. In the examples herein, initiating the cuts at radially outwards positions determines one end of the cuts, and the intersection of the cuts, typically at a radially central portion of the multi-cuspid valvular shunt, determines the other end, and reduces and/or eliminates the dependence of the lengths of the cuts on material properties of the septal wall tissue, angle of the cutting tool, e.g., inner member 102, or the cutting action, e.g., how fast or slow the cut is made.

In some examples, the clinician may cause a proximal and/or distal support structure to support a proximal and/or distal surface of at least a portion of septal wall 14 along the length of one or more of the cuts. The proximal and/or distal structure may be configured to reduce movement of septal wall 14 in the proximal direction while initiating the cuts and/or while cutting septal wall 14. In some examples, the proximal and/or distal structure may be configured to reduce stretching and/or compression of septal wall 14 tissue along the cut while initiating the cuts and/or while cutting septal wall 14.

The clinician may cut septal wall 14 from the radially outwards position to a position on septal wall 14 that is at or substantially near an intersection of at least two of the cuts (204). For example, the clinician may cut septal wall 14 from the first end (at positions 156) of the cuts to the second ends of the cuts (at the intersection point, puncture point and/or opening 16, and/or a radially central portion of a multi-cuspid shunt) to form at least a portion of the multi-cuspid valvular shunt, e.g., via cutting members 134 in the deployed and/or expanded configuration. The expandable members 124 may be circumferentially positioned about elongated support member 112 every 120 degrees, e.g., three expandable members 124 may be evenly spaced about elongated member 112 such that multi-cuspid valvular shunt 402 is formed. In other examples, inner member 102 may include two, three, four or five expandable members such that the clinician may form multi-cuspid valvular shunts 404-410, respectively, after cutting the septal tissue.

In some examples, the clinician may retract distal portion 116 and/or inner member 102 towards the proximal side of septal wall 14 to cut septal wall 14 from the first, radially outwards end of the cut to the second, radially central end of the cut at or near the intersection of two or more cuts, e.g., via the cutting surface. In some examples, the clinician may retract cutting elements 134 radially inwards towards longitudinal axis 166 of distal portion 116 of inner member 102 to cut septal wall 14 from the first, radially outwards ends of the cuts to the second, radially central ends of the cuts at or near the intersection of two or more cuts. For example, cutting elements 134 are angled with respect to longitudinal axis 166, and proximal motion of the cutting elements may cause the cutting elements to cut septal wall 14 in a radially inwards direction from positions 156 to the intersection point, e.g., at or near longitudinal axis 166. In some examples, the clinician may retract cutting elements 134 radially inwards towards longitudinal axis 166 of distal portion 116 of inner member 102 to cut septal wall 14 from the first, radially outwards ends of the cuts to the second, radially central ends of the cuts at or near the intersection of two or more cuts while also retracting distal portion 116 and/or inner member 102 towards the proximal side of septal wall 14. For example, the clinician may cause cutting elements to cut septal wall 14 by proximally retracting inner member 102 and causing expandable members 124 to retract radially inwards to cut septal wall 14 via “scissors” like action. In some examples, the clinician may initiate two or more cuts, e.g., at (202) and/or cut two or more cuts, e.g., at (204), substantially concurrently.

In some examples, the clinician may fully retract cutting elements 134 radially inwards towards longitudinal axis 166 of distal portion 116 of inner member 102, e.g., to be at least partially within slots 131, such that cutting points 146 and/or cutting surfaces 144 are not exposed to tissue of the patient after cutting the septal wall to the second end of the cut.

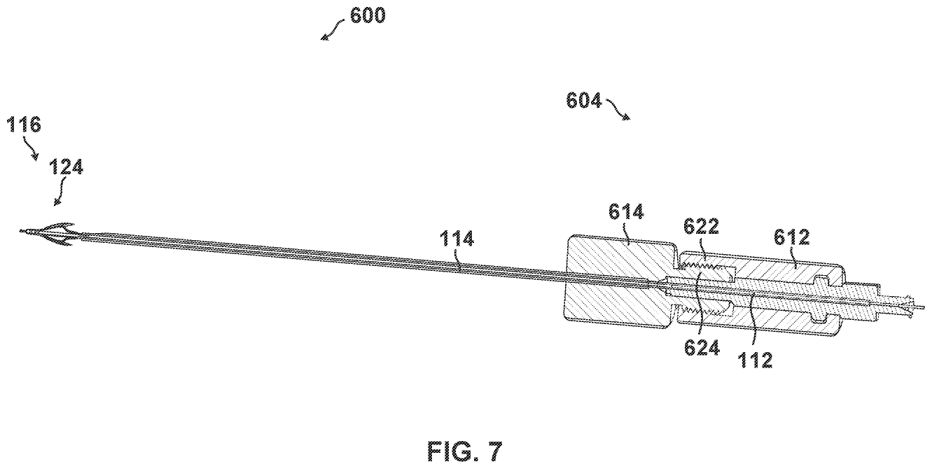

FIG. 7 is a schematic cross-sectional view depicting an example medical system 600 including an example deployment mechanism 604. Medical system 600 may be substantially similar to medical system 100 of FIGS. 1-3 and including deployment mechanism 604. In the example shown, deployment mechanism 604 includes a first portion 612 that may be integral with and/or attached to elongated support member 112. Deployment mechanism 604 includes a second portion 614 that may be integral with and/or attached to movable member 114. Second portion 614 comprises a movable shaft including threads 624, and first portion 612 comprises a movable shaft including threads 622. Upon rotation of first portion 612 in a first direction relative to second portion 614, first portion 612 and elongated support member 112 are configured to move in an axial direction, e.g., along the longitudinal length of movable member 114 and elongated support member 112, in a distal direction relative to movable member 114, thereby causing expandable members 124 to retract to the undeployed configuration. Upon rotation of first portion 612 in a second direction opposite the first direction relative to second portion 614, first portion 612 and elongated support member 112 are configured to move in an axial direction, e.g., along the longitudinal length of movable member 114 and elongated support member 112, in a proximal direction relative to movable member 114, thereby causing expandable members 124 to expand to the deployed configuration, as shown in FIG. 7. In other examples, (not shown), medical system 100 and/or 600 may include at least one wire attached to elongated support member 112 and configured to proximally move elongated support member 112 relative to movable member 114 to cause expandable members 124 to expand to the deployed configuration. The at least one wire may be configured to release the elongated support member 112 to distally move relative to the movable member 114, e.g., via elasticity of expandable members 124, to cause expandable members 124 to retract to the undeployed configuration.

Accordingly, although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.

Example 1: A medical device includes: an elongated support member defining a longitudinal axis; and a plurality of expandable members at a distal portion of the elongated support member, wherein the plurality of expandable members are positioned circumferentially about the elongated support member, wherein at least a portion of each of the expandable members is configured to radially extend from the elongated support member, wherein, to form a multi-cuspid valvular shunt, each expandable member of the plurality of expandable members comprises a respective cutting element of a plurality of elements, each cutting element of the plurality of cutting elements comprising a cutting surface facing towards the longitudinal axis and configured to initiate a respective cut of a plurality of cuts of a septal wall tissue at a first end of the respective cut and to cut the septal wall tissue from the first end to a second end of the respective cut comprising an intersection of the plurality of cuts.

Example 2: The medical device of example 1, wherein the intersection comprises a radially central portion of the multi-cuspid valvular shunt.

Example 3: The medical device of any one of examples 1 and 2 or any of examples 1 and 2, wherein each cutting element of the plurality of cutting elements is configured to determine a respective length of the respective cut by initiating the respective cut at the first end.

Example 4: The medical device of any one of examples 1-3, wherein each cutting element of the plurality cutting elements is configured initiate the respective cut at the first end of the respective cut to cause the respective length of the respective cut to be independent of at least one of a thickness variation of the septal wall, a density variation of the septal wall, a tension of the septal wall, or an angle of a cutting tool used to make the respective cut relative to a surface of the septal wall.

Example 5: The medical device of any one of examples 1-4, further includes a proximal support structure configured to support a proximal surface of at least a portion of the septal wall along each length of a plurality of lengths of the plurality of cuts and configured to reduce movement of the at least the portion of the septal wall in the proximal direction while the cutting surfaces are cutting the septal wall.

Example 6: The medical device of example 5, further includes a distal support structure configured to support a distal surface of at least a portion of the septal wall along each length of a plurality of lengths of the plurality of cuts and configured to at least one of reduce movement of the at least the portion of the septal wall in the distal direction while the cutting surfaces are cutting the septal wall or reduce a stretching of the septal wall along the cuts while the cutting surfaces are cutting the septal wall.

Example 7: The medical device of example 6, wherein at least one of the proximal support structure or the distal support structure comprises at least one of an expandable balloon or an expandable frame.

Example 8: The medical device of any one of examples 1-7, wherein each expandable member of the plurality of expandable members is configured to prevent the respective cutting surface from contacting tissue of the patent when the expandable member is radially retracted to the elongated support member.

Example 9: The medical device of any one of examples 1-8, wherein the plurality of expandable members is configured to initiate the plurality of cuts substantially concurrently and cut septal wall tissue substantially concurrently.

Example 10: A method includes: initiating a cut of a septal wall tissue between a right atrium and left atrium of a heart of a patient at a first end of the cut, wherein the cut comprises a second end at an intersection of two or more cuts of a multi-cuspid valvular shunt; and cutting the septal wall from the first end of the cut to the second end of the cut to form a portion of the multi-cuspid valvular shunt.

Example 11: The method of example 10, wherein the intersection comprises a radially central portion of the multi-cuspid valvular shunt.

Example 12: The method of any one of examples 10 and 11 or any of examples 10 and 11, wherein initiating the cut at the first end of the cut determines the length of the cut.

Example 13: The method of any one of examples 10-12, wherein initiating the cut at the first end of the cut causes the length of the cut to be independent of at least one of a thickness variation of the septal wall, a density variation of the septal wall, a tension of the septal wall, or an angle of a cutting tool used to make the cut relative to a surface of the septal wall.

Example 14: The method of any one of examples 10-13, further includes supporting a proximal surface of at least a portion of the septal wall along the length of the cut with a structure configured to reduce movement of the at least the portion of the septal wall in the proximal direction while cutting the septal wall.

Example 15: The method of example 14, further includes supporting a distal surface of the least the portion of the septal wall along the length of the cut with a structure configured to at least one of reduce movement of the at least the portion of the septal wall the distal direction while cutting the septal wall or reduce a stretching of the septal wall along the cut while cutting the septal wall.

Example 16: The method of any one of examples 10-15, further includes puncturing the septal wall with a cutting tool in an undeployed configuration, wherein the puncture comprises the intersection of the two or more cuts of the multi-cuspid valvular shunt; advancing a distal portion of the cutting tool through the puncture to a distal side of the septal wall; and expanding a cutting element radially outwards from a longitudinal axis of the distal portion of the cutting tool to a deployed configuration on the distal side of the septal wall, wherein expanding the cutting element exposes a cutting surface of the cutting element, wherein the cutting surface of the cutting element faces towards the longitudinal axis of the distal portion of the cutting tool.

Example 17: The method of example 16, further comprising retracting the distal portion towards the proximal side of the septal wall to cut the septal wall from the first end of the cut to the second end of the cut via the cutting surface.

Example 18: The method of example 17, further comprising further comprising retracting the cutting element radially inwards towards the longitudinal axis of the distal portion of the cutting tool to cut the septal wall from the first end of the cut to the second end of the cut while retracting the distal portion towards the proximal side of the septal wall.

Example 19: The method of any one of examples 17 and 18 or any of examples 17 and 18, further comprising fully retracting the cutting element radially inwards towards the longitudinal axis of the distal portion of the cutting tool such that the cutting surface is not exposed to tissue of the patient after cutting the septal wall to the second end of the cut.

Example 20: The method of any one of examples 16-19, further includes retracting the distal portion towards the proximal side of the septal wall to initiate the cut at the first end of the cut; and retracting the cutting element radially inwards towards the longitudinal axis of the distal portion of the cutting tool to cut the septal wall from the first end of the cut to the second end of the cut.

Example 21: The method of any one of examples 10-20, wherein the cut is a first cut and the portion of the multi-cuspid valvular shunt is a first portion of the multi-cuspid valvular shunt, the method further includes initiating a second cut of a septal wall between a right atrium and left atrium of a heart of a patient at a first end of the second cut, wherein the second cut comprises a second end at the intersection of the multi-cuspid valvular shunt; and cutting the septal wall from the first end of the second cut to the second end of the second cut to form a second portion of the multi-cuspid valvular shunt.

Example 22: The method of any one of examples 10-21, wherein the first cut and the second cut occur substantially concurrently.

Example 23: A medical system includes: a catheter defining a lumen; an inner member configured to be received in the catheter lumen and extend distally outward from a distal opening of the catheter, wherein the inner member comprises: an elongated support member configured to move axially within the catheter lumen, the elongated support member defining a longitudinal axis; and a plurality of expandable members at a distal portion of the elongated support member, wherein the plurality of expandable members are positioned circumferentially about the elongated support member, wherein at least a portion of each of the expandable members is configured to radially extend from the elongated support member, wherein, to form a multi-cuspid valvular shunt, each expandable member of the plurality of expandable members comprises a respective cutting element of a plurality of elements, each cutting element of the plurality of cutting elements comprising a cutting surface facing towards the longitudinal axis and configured to initiate a respective cut of a plurality of cuts of a septal wall tissue at a first end of the respective cut and to cut the septal wall tissue from the first end to a second end of the respective cut comprising an intersection of the plurality of cuts.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A medical device comprising:
an elongated support member defining a longitudinal axis; and
a plurality of expandable members at a distal portion of the elongated support member, wherein the plurality of expandable members are positioned circumferentially about the elongated support member,
wherein at least a portion of each of the expandable members is configured to radially extend from the elongated support member,
wherein, to form a multi-cuspid valvular shunt, each expandable member of the plurality of expandable members comprises a respective cutting element of a plurality of elements, each cutting element of the plurality of cutting elements comprising a cutting surface facing towards the longitudinal axis and configured to initiate a respective cut of a plurality of cuts of a septal wall tissue at a first end of the respective cut and to cut the septal wall tissue from the first end to a second end of the respective cut, wherein the second end of the respective cut comprises an intersection of the plurality of cuts.

2. The medical device of claim 1, wherein the intersection comprises a radially central portion of the multi-cuspid valvular shunt.

3. The medical device of claim 1, wherein each cutting element of the plurality of cutting elements is configured to determine a respective length of the respective cut by initiating the respective cut at the first end.

4. The medical device of claim 1, wherein each cutting element of the plurality cutting elements is configured initiate the respective cut at the first end of the respective cut to cause the respective length of the respective cut to be independent of at least one of a thickness variation of the septal wall, a density variation of the septal wall, a tension of the septal wall, or an angle of a cutting tool used to make the respective cut relative to a surface of the septal wall.

5. The medical device of claim 1, further comprising:
a proximal support structure configured to support a proximal surface of at least a portion of the septal wall along each length of a plurality of lengths of the plurality of cuts and configured to reduce movement of the at least the portion of the septal wall in the proximal direction while the cutting surfaces are cutting the septal wall.

6. The medical device of claim 5, further comprising:
a distal support structure configured to support a distal surface of at least a portion of the septal wall along each length of a plurality of lengths of the plurality of cuts and configured to at least one of reduce movement of the at least the portion of the septal wall in the distal direction while the cutting surfaces are cutting the septal wall or reduce a stretching of the septal wall along the cuts while the cutting surfaces are cutting the septal wall.

7. The medical device of claim 6, wherein at least one of the proximal support structure or the distal support structure comprises at least one of an expandable balloon or an expandable frame.

8. The medical device of claim 1, wherein each expandable member of the plurality of expandable members is configured to prevent the respective cutting surface from contacting tissue of the patent when the expandable member is radially retracted to the elongated support member.

9. The medical device of claim 1, wherein the plurality of expandable members is configured to initiate the plurality of cuts substantially concurrently and cut septal wall tissue substantially concurrently.

10. A medical system comprising:
a catheter defining a lumen;
an inner member configured to be received in the catheter lumen and extend distally outward from a distal opening of the catheter, wherein the inner member comprises:
an elongated support member configured to move axially within the catheter lumen, the elongated support member defining a longitudinal axis; and
a plurality of expandable members at a distal portion of the elongated support member, wherein the plurality of expandable members are positioned circumferentially about the elongated support member, wherein at least a portion of each of the expandable members is configured to radially extend from the elongated support member,
wherein, to form a multi-cuspid valvular shunt, each expandable member of the plurality of expandable members comprises a respective cutting element of a plurality of elements, each cutting element of the plurality of cutting elements comprising a cutting surface facing towards the longitudinal axis and configured to initiate a respective cut of a plurality of cuts of a septal wall tissue at a first end of the respective cut and to cut the septal wall tissue from the first end to a second end of the respective cut comprising an intersection of the plurality of cuts.

11. A method comprising:
initiating a cut of a septal wall tissue between a right atrium and left atrium of a heart of a patient at a first end of the cut, wherein the cut comprises a second end at an intersection of two or more cuts of a multi-cuspid valvular shunt; and
cutting the septal wall from the first end of the cut to the second end of the cut to form a portion of the multi-cuspid valvular shunt.

12. The method of claim 11, wherein the intersection comprises a radially central portion of the multi-cuspid valvular shunt.

13. The method of claim 11, wherein initiating the cut at the first end of the cut determines the length of the cut.

14. The method of claim 11, wherein initiating the cut at the first end of the cut causes the length of the cut to be independent of at least one of a thickness variation of the septal wall, a density variation of the septal wall, a tension of the septal wall, or an angle of a cutting tool used to make the cut relative to a surface of the septal wall.

15. The method of claim 11, further comprising:
supporting a proximal surface of at least a portion of the septal wall along the length of the cut with a structure configured to reduce movement of the at least the portion of the septal wall in the proximal direction while cutting the septal wall.

16. The method of claim 15, further comprising:
supporting a distal surface of the least the portion of the septal wall along the length of the cut with a structure configured to at least one of reduce movement of the at least the portion of the septal wall the distal direction while cutting the septal wall or reduce a stretching of the septal wall along the cut while cutting the septal wall.

17. The method of claim 11, further comprising:
puncturing the septal wall with a cutting tool in an undeployed configuration, wherein the puncture comprises the intersection of the two or more cuts of the multi-cuspid valvular shunt;
advancing a distal portion of the cutting tool through the puncture to a distal side of the septal wall; and
expanding a cutting element radially outwards from a longitudinal axis of the distal portion of the cutting tool to a deployed configuration on the distal side of the septal wall, wherein expanding the cutting element exposes a cutting surface of the cutting element, wherein the cutting surface of the cutting element faces towards the longitudinal axis of the distal portion of the cutting tool.

18. The method of claim 17, further comprising retracting the distal portion towards the proximal side of the septal wall to cut the septal wall from the first end of the cut to the second end of the cut via the cutting surface.

19. The method of claim 18, further comprising retracting the cutting element radially inwards towards the longitudinal axis of the distal portion of the cutting tool to cut the septal wall from the first end of the cut to the second end of the cut while retracting the distal portion towards the proximal side of the septal wall.

20. The method of claim 18, further comprising fully retracting the cutting element radially inwards towards the longitudinal axis of the distal portion of the cutting tool such that the cutting surface is not exposed to tissue of the patient after cutting the septal wall to the second end of the cut.

21. The method of claim 17, further comprising:
retracting the distal portion towards the proximal side of the septal wall to initiate the cut at the first end of the cut; and
retracting the cutting element radially inwards towards the longitudinal axis of the distal portion of the cutting tool to cut the septal wall from the first end of the cut to the second end of the cut.

22. The method of claim 11, wherein the cut is a first cut and the portion of the multi-cuspid valvular shunt is a first portion of the multi-cuspid valvular shunt, the method further comprising:
initiating a second cut of a septal wall between a right atrium and left atrium of a heart of a patient at a first end of the second cut, wherein the second cut comprises a second end at the intersection of the multi-cuspid valvular shunt; and
cutting the septal wall from the first end of the second cut to the second end of the second cut to form a second portion of the multi-cuspid valvular shunt.

23. The method of claim 11, wherein the first cut and the second cut occur substantially concurrently.
